(19) United States
(12) Patent Application Publication
(10) Pub. No.: US 2015/0366513 A1
(43) Pub. Date: Dec. 24, 2015

(54) PULSE WAVE SENSOR

(71) Applicant: Rohm Co., Ltd., Tokyo (JP)

(72) Inventor: Koji Terumoto, Kyoto (JP)

(21) Appl. No.: 14/744,571

(22) Filed: Jun. 19, 2015

(30) Foreign Application Priority Data
Jun. 24, 2014 (JP) 2014129390

(51) Int. Cl. A61B 5/00; A61B 5/024

(52) U.S. Cl. CPC A61B 5/7225; A61B 5/02427

(57) ABSTRACT

A pulse wave sensor includes: an optical sensor unit configured to irradiate a living body with light emitted from a light emitting unit and detect light reflected from or transmitted through the living body with a light receiving unit to generate a current signal in accordance with a light reception intensity; a pulse driving unit configured to turn on or turn off the light emitting unit at a predetermined frame frequency and a predetermined duty rate; a current-voltage conversion circuit configured to convert the current signal into a voltage signal; and a detection circuit configured to extract an upper envelope and a lower envelope of the voltage signal and obtain a difference therebetween to generate a detection signal.

### CROSS-REFERENCE TO RELATED APPLICATION

This application is based upon and claims the benefit of priority from Japanese Patent Application No. 2014-129390, filed on Jun. 24, 2014, the entire contents of which are incorporated herein by reference.

### TECHNICAL FIELD

The present disclosure relates to a pulse wave sensor.

### BACKGROUND

A pulse wave sensor (a so-called pulse wave sensor of a photoelectric type) irradiates a living body (e.g., an examinee's arm or finger, etc.) with light emitted from a light emitting unit and detects a pulse wave of the examinee based on a light reception intensity of light transmitted through the living body. A pulse wave sensor of this type is capable of obtaining various kinds of pulse wave information (e.g., a pulse rate of the examinee, etc.) based on the characteristics of a pulse wave signal (e.g., a fluctuation period of a pulse wave signal, etc.) corresponding to the light reception intensity, since the light reception intensity varies with the examinee's pulse wave.

However, when a pulse wave measurement (e.g., a pulse rate measurement) is made during an examinee's activities in a harsh sunshiny outdoor environment (e.g., on a sunny day where the illuminance is about 100,000 lux), the measured signal is saturated due to disturbance from ambient light.

### SUMMARY

The present disclosure provides a pulse wave sensor capable of accurately measuring a pulse wave (i.e., measuring a beat) even in an outdoor activities environment.

According to one embodiment of the present disclosure, there is provided a pulse wave sensor including an optical sensor unit configured to irradiate a living body with light emitted from a light emitting unit and detect light reflected from or light transmitted through the living body with a light receiving unit to generate a current signal corresponding to a light reception intensity, a pulse driving unit configured to turn on or turn off the light emitting unit at a predetermined frame frequency and a predetermined duty ratio, a current/voltage conversion circuit configured to convert the current signal into a voltage signal, and a detection circuit configured to respectively extract an upper envelope and a lower envelope of the voltage signal and obtain a difference therebetween to generate a detection signal.

In addition, according to the embodiment of the present disclosure, the detection circuit may include an upper envelope detector unit to extract an upper envelope signal of the voltage signal, a lower envelope detector unit to extract a lower envelope signal of the voltage signal, and a differential amplification unit to obtain a difference between the lower envelope signal and the upper envelope signal to generate the detection signal.

In addition, according to the embodiment of the present disclosure, the upper envelope detector unit may include a first diode including an anode connected to an input terminal of the voltage signal and a cathode connected to an output terminal of the upper envelope signal, and a first capacitor including a first terminal connected to an output terminal of the upper envelope signal and a second terminal connected to a terminal for applying a reference voltage.

In addition, according to the embodiment of the present disclosure, the lower envelope detector unit may include a second diode including a cathode connected to an input terminal of the voltage signal and an anode connected to an output terminal of the lower envelope signal, and a second capacitor including a first terminal connected to an output terminal of the lower envelope signal and a second terminal connected to a terminal for applying a reference voltage.

In addition, according to the embodiment of the present disclosure, the detection circuit may further include a level shifter unit to adjust each signal level of the upper envelope signal and the lower envelope signal respectively match an input range of the differential amplification unit.

In addition, according to the embodiment of the present disclosure, the level shifter unit may be a high pass filter.

In addition, according to the embodiment of the present disclosure, the current/voltage conversion unit may be a trans-impedance amplifier.

In addition, according to the embodiment of the present disclosure, the pulse wave sensor may further include a band pass filter circuit configured to remove both low-frequency components and high-frequency components superimposed on the detection signal to generate a filter signal.

In addition, according to the embodiment of the present disclosure, the pulse wave sensor may further include an amplifier circuit configured to amplify the filter signal by a predetermined gain to generate an output signal.

In addition, according to the embodiment of the present disclosure, a wavelength output from the light emitting unit may fall within a visible light region where a wavelength is 600 nm or smaller.

### BRIEF DESCRIPTION OF THE DRAWINGS

FIG. 1 shows a schematic diagram to explain principles of measuring pulse waves from a wrist.

FIG. 2 shows a wave form diagram illustrating a change of attenuation of light (i.e., a light absorption level) within a living body with time.

FIG. 3 shows a block diagram illustrating an exemplary configuration of a pulse wave sensor 1.

FIG. 4 shows a circuit diagram illustrating an exemplary configuration of an optical sensor unit 11 and a pulse driving unit 17.

FIG. 5 shows a block diagram illustrating an exemplary configuration of a filter unit 12.

FIG. 6 shows a circuit diagram illustrating an exemplary configuration of a transimpedance amplifier 121.

FIG. 7 shows a circuit diagram illustrating an exemplary configuration of a detection circuit 123.

FIG. 8 shows a wave form diagram illustrating an example of a voltage signal Sa.

FIG. 9 shows a wave form diagram illustrating examples of an upper envelope signal SU and a lower envelope signal SL.

FIG. 10 shows a wave form diagram illustrating an example of an output signal Se.

FIG. 11A and FIG. 11B show views illustrating a first measurement example (i.e., a detection of only a lower envelope) taken in an outdoor environment.

FIG. 12A and FIG. 12B show views illustrating a second measurement example (i.e., a detection of a difference between an upper envelope and a lower envelope) taken in an outdoor environment.

### DETAILED DESCRIPTION

Embodiments of the present disclosure will now be described in detail with reference to the drawings. Throughout the drawings, the same or similar elements, members and processes are denoted by the same reference numerals and explanation of which will not be repeated. The disclosed embodiments are provided for the purpose of illustration, not limitation, of the present disclosure and all features and combinations thereof described in the embodiments cannot be necessarily construed to describe the spirit of the present disclosure.

### <Principles of Pulse Wave Measurement>

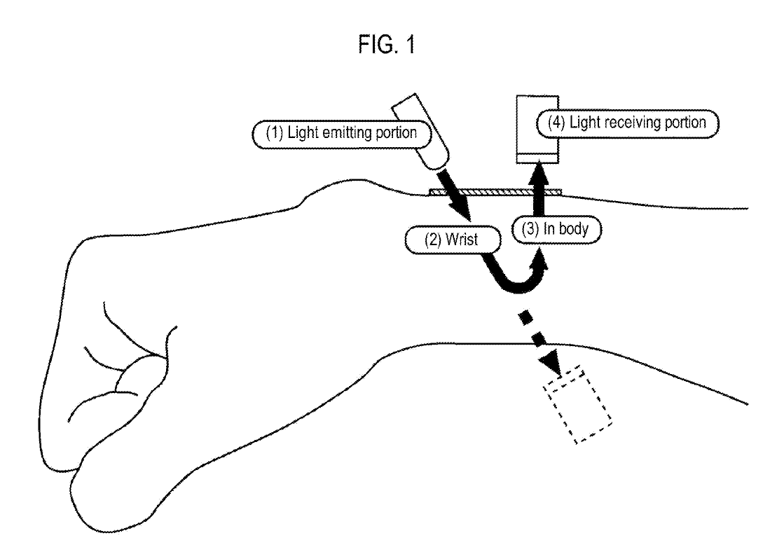

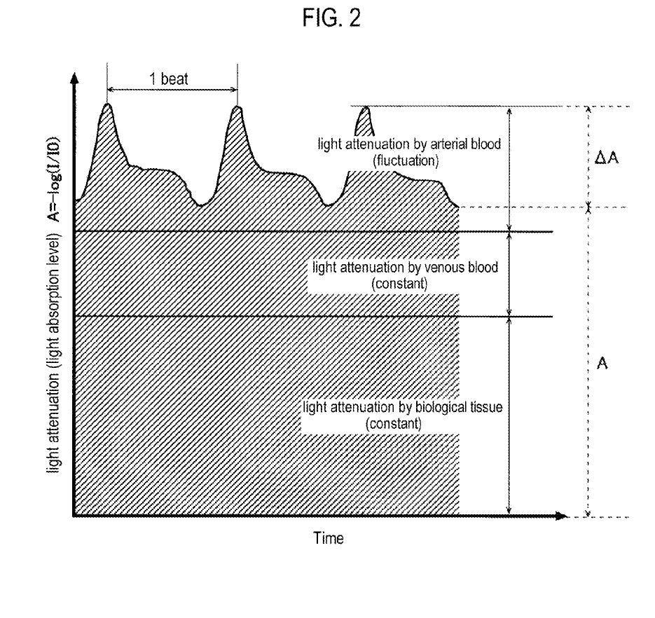

FIG. 1 shows a schematic diagram to explain the principles of a pulse wave measurement of a wrist. FIG. 2 shows a wave form diagram illustrating a situation where a light attenuation amount (i.e., a light absorption level) within a living body changes with time.

In a pulse wave measurement using a plethysmography method, for example, as shown in FIG. 1, light from a light emitting unit (e.g., a light emitting diode (LED), etc.) is irradiated on a portion of a living body (i.e., the wrist shown in FIG. 1) against which a measurement window is pressed. Then, the intensity of the light that has been transmitted through and out of the living body is detected with a light receiving unit (e.g., a photo diode, a photo transistor, etc.). Herein, as shown in FIG. 2, while the light attenuation amount (i.e., the light absorption level) due to biological tissues or venous blood (i.e., deoxyhemoglobin Hb) is constant, the light attenuation amount (i.e., the light absorption level) due to arterial blood (i.e., oxyhemoglobin HbO₂) may fluctuate with time according to an examinee's pulse. Thus, by measuring the change in the light absorption level of peripheral arteries using a “living body window” (i.e., a wavelength region where light is easily transmitted through a living body), which ranges from a visible light region to a near infrared region, it is possible to measure the volume pulse in a non-invasive manner.

While FIG. 1 shows a pulse wave sensor (a light emitting unit and a light receiving unit) as being mounted on the dorsal side (i.e., the outer side) of the wrist for convenience of illustration, there is no intention to limit the mounting position of the pulse wave sensor to this position. The pulse wave sensor may be mounted on the ventral side (i.e., the inner side) of a wrist or other parts of a living body (e.g., a fingertip, the third joint of a finger, a forehead, the glabella, the tip of the nose, cheeks, under eyes, a temple, an ear lobe, etc.).

### <Information Obtainable From a Pulse Wave>

In addition, a pulse wave, which is controlled by the heart or the autonomic nerve system, does not always exhibit a constant behavior, but will show a variety of variations (i.e., fluctuations) according to the state of an examinee. Accordingly, a variety of body information of the examinee can be acquired by analyzing the changes (i.e., fluctuations) in the pulse wave. For example, the athletic ability or tension of the examinee can be learned from the examinee's heart rate. The fatigue level, the deep sleep level, and the stress level of the examinee, etc. can be learned from the fluctuations in the examinee's heart rate. Furthermore, the blood vessel age or arterial stiffness of the examinee, etc. can be learned based on the acceleration pulse wave acquired by differentiating the pulse wave two times along the time axis.

### <Pulse Wave Sensor>

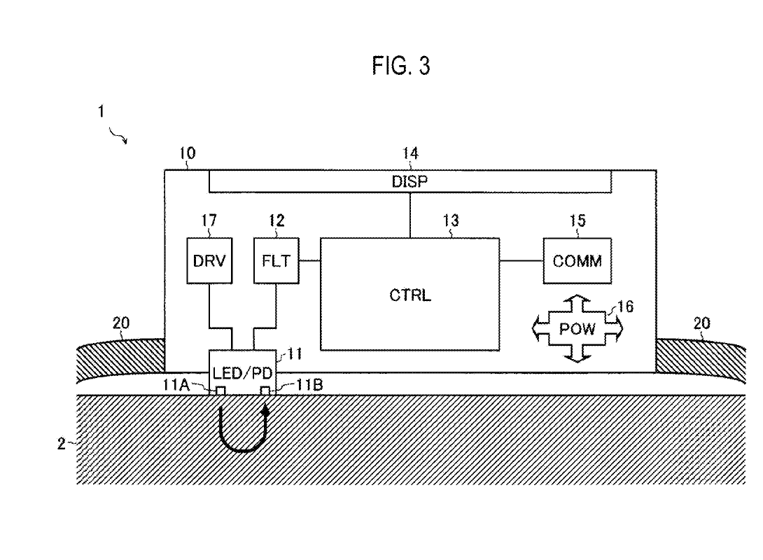

FIG. 3 shows a block diagram illustrating an exemplary configuration of a pulse wave sensor. The pulse wave sensor 1 of the exemplary configuration has a bracelet structure (i.e., a wristwatch structure) with a main body 10 and a belt 20, the belt 20 being attached to both ends of the main body 10 and worn on the living body 2 (specifically, the wrist). As a material of the belt 20, leather, metal, resin, etc. may be used.

The main body 10 includes an optical sensor unit 11, a filter unit 12, a control unit 13, a display unit 14, a communication unit 15, a power supply unit 16, and a pulse driving unit 17.

The optical sensor unit 11 is provided on a rear surface of the main body 10 (a surface facing the living body 2). The optical sensor unit 11 generates a current signal corresponding to a light reception intensity by irradiating the living body 2 with light emitted from a light emitting unit 11A and detecting light reflected from the living body 2 (or, possibly light transmitted through the living body 2) with a light receiving unit 11B. In the pulse wave sensor 1 of the exemplary configuration, the optical sensor unit 11 is not of a configuration in which the light emitting unit 11A and the light receiving unit 11B are provided on opposite sides with the living body 2 interposed therebetween (i.e., a so-called transmission type, see a dashed line arrow in FIG. 1), but is of a configuration in which the light emitting unit 11A and the light receiving unit 11B are provided on the same side with respect to the living body 2 (i.e., a so-called reflection type, see solid line arrows in FIG. 1). In addition, the present inventors have confirmed experimentally that a pulse wave measurement in the wrist is sufficiently practicable.

The filter unit 12 performs a variety of signal processing (e.g., a current/voltage conversion processing, detection processing, filtering, and amplification processing) on a current signal input from the optical sensor unit 11 and outputs it to the control unit 13. In addition, the specific configuration of the filter unit 12 will be described later in detail.

The control unit 13 generally controls the entire operation of the pulse wave sensor 1. Further, the control unit 13 obtains various kinds of information (e.g., a fluctuation of a pulse wave, a heart rate, a fluctuation of the heart rate, and an acceleration pulse wave, etc.) related to a pulse wave by performing a variety of signal processing on an output signal from the filter unit 12. In addition, it is possible to suitably use a central processing unit (CPU), etc. as the control unit 13.

The display unit 14 is provided on a surface of the main body 10 (a surface that does not face the living body 2) to output display information (e.g., information related to date or time, and other information including measurement results of the pulse wave). In other words, the display unit 14 is comparable to a letter board surface of a wristwatch. In addition, it is possible to suitably use a liquid crystal display panel, etc. as the display unit 14.

The communication unit 15 transmits measurement data of the pulse wave sensor 1 to an external device (e.g., a personal computer or a cellular phone, etc.) by wire or wirelessly. In particular, in a configuration in which measurement data of the pulse wave sensor 1 is to be wirelessly transmitted to the external device, it is unnecessary to connect the pulse wave sensor 1 and the external device by wire, and thus, for example, it is possible to perform a real-time transmission of measurement data without limiting the behavior of an examinee. Also, when the pulse wave sensor 1 has a waterproof structure, it is desirable to employ a wireless transmission method as a method for an external transmission of measurement data, since it is desirable to completely eliminate external terminals. When employing a wireless transmission method, it is possible to suitably use a Bluetooth ® wireless communication module IC as the communication unit 15.

The power supply unit 16 includes a battery and a DC/DC converter. The power supply unit 16 converts an input voltage from the battery to a desired output voltage and supplies it to each unit of the pulse wave sensor 1. In this manner, in cases where the pulse wave sensor 1 is driven by a battery, it is unnecessary to connect the pulse wave sensor 1 with a power supply cable to an outside source for the pulse wave measurement. Thus, without limiting the behavior of the examinee, it is possible to perform the pulse wave measurement. In addition, it is possible to use a rechargeable secondary battery such as a lithium ion secondary battery or an electric double layer capacitor as the above-mentioned battery. As such, when using a secondary battery as the battery of the pulse wave sensor 1, it is possible to improve the convenience of the pulse wave sensor 1 since a battery need not be replaced. In addition, when charging a battery, a contact power feeding method using a universal serial bus (USB) cable, etc. or a non-contact power feeding method (such as, an electromagnetic induction method, an electric field coupling method, and a magnetic field resonance method) may be used as a method for supplying power from an external source. However, when the pulse wave sensor 1 has a waterproof structure, it is desirable to employ a non-contact power feeding method such as a method for supplying power from an external source, since it is desirable to completely eliminate external terminals.

The pulse driving unit 17 turns on or turns off the light emitting unit 11A of the optical sensor unit 11 at a predetermined frame frequency f (e.g., 50 to 1000 Hz) and a predetermined duty ratio D (e.g., ⅛ to 1/200).

As described above, if the pulse wave sensor 1 has a bracelet structure, there is almost no concern about the pulse wave sensor 1 coming off from the wrist during the measurement of a pulse wave unless the examinee purposely removes the pulse wave sensor 1 from the wrist. Thus, it is possible to perform the pulse wave measurement without limiting the behavior of the examinee.

In addition, if the pulse wave sensor 1 has a bracelet structure, the measurement can be made without the examinee being overly conscious of the pulse wave sensor 1. Thus, the measurement may be done without causing excessive stress to the examinee, even in the case where a continuous pulse wave measurement is made over a long period of time (e.g., several days to several months).

Especially, if the pulse wave sensor 1 has a display unit 14 to display date or time information as well as measurement results of the pulse wave (i.e., if pulse wave sensor 1 has a wristwatch structure), the examinee can routinely wear the pulse wave sensor 1 as a wristwatch. Thus, any feeling of resistance against wearing the pulse wave sensor 1 can be eliminated, and furthermore, it may contribute to developing new consumers.

Moreover, it is desirable that the pulse wave sensor 1 has a water-proof structure. With such a configuration, it is possible to measure a pulse wave without the pulse wave sensor 1 breaking down even when the pulse wave sensor 1 is soaked in water (e.g., rain) or sweat. Furthermore, if the pulse wave sensor 1 is shared by many persons (e.g., when used as a rental at a sports gym), the pulse wave sensor 1 can be kept clean by washing the whole pulse wave sensor 1.

### <Optical Sensor Unit and Pulse Driving Unit>

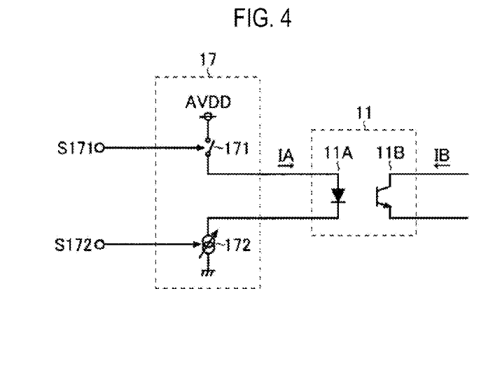

FIG. 4 shows a circuit diagram illustrating an exemplary configuration of an optical sensor unit 11 and a pulse driving unit 17. The optical sensor unit 11 of the exemplary configuration includes a light emitting diode 11A (corresponding to a light emitting unit) and a phototransistor 11B (corresponding to a light receiving unit). The pulse driving unit 17 of the exemplary configuration includes a switch 171 and a current source 172.

An anode of the light emitting diode 11A is connected to a terminal for applying a power supply voltage AVDD via the switch 171. A cathode of the light emitting diode 11A is connected to a ground terminal via a current source 172. The switch 171 is turned on or turned off according to a pulse driving signal S171. The current source 172 generates a constant current IA according to a brightness control signal S172. In order to perform a pulse wave measurement with high accuracy during physical exercise or in an outdoor environment, it is desirable to pulse drive a light emitting diode 11A with brightness as high as possible.

If the switch 171 is turned on, a current path through which the constant current IA flows is formed and the light emitting diode 11A is turned on. Thus, light from the light emitting diode 11A is irradiated to the living body 2. At this time, a current signal IB generated according to the light reception intensity of a reflected light returned from the living body 2 flows between a collector and an emitter of the phototransistor 11B. Meanwhile, if the switch 171 is turned off, the current path through the constant current IA flows is cut off and the light emitting diode 11A is turned off.

### <Filter Unit>

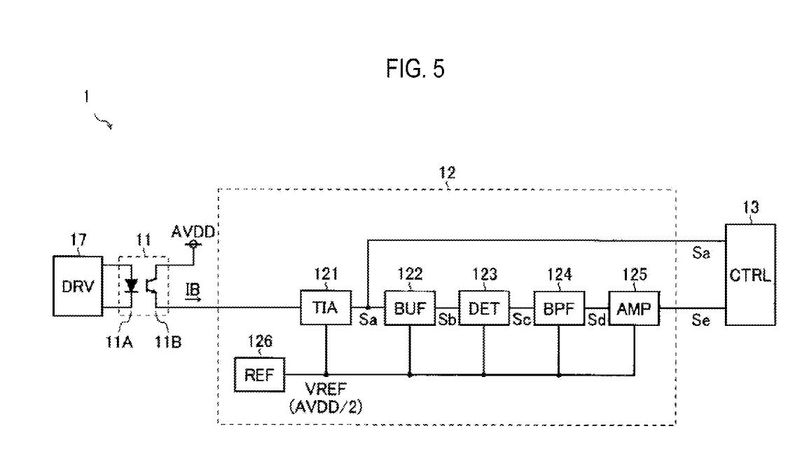

FIG. 5 shows a block diagram illustrating an exemplary configuration of a filter unit 12. The filter unit 12 of the exemplary configuration includes a trans-impedance amplifier 121 (hereinafter, abbreviated as TIA 121), a buffer circuit 122, a detection circuit 123, a band pass filter circuit 124, an amplifier circuit 125, and a reference voltage generation circuit 126.

The TIA 121 of the exemplary configuration is a type of a current/voltage conversion circuit to convert a current signal IB to a voltage signal Sa and output it to the buffer circuit 122 and the control unit 13 of a subsequent stage.

The buffer circuit 122 is a voltage follower to transfer the voltage signal Sa to a subsequent stage as a buffer signal Sb.

The detection circuit 123 generates a detection signal Sc by extracting only an envelope of the voltage signal Sb, which is pulse-driven, and outputs it to a subsequent stage. More specifically, the detection circuit 123 generates the detection signal Sc by extracting the upper envelope (i.e., the signal changes at the upper end side of pulses) and the lower envelope (i.e., the signal changes at the lower end side of pulses) of the voltage signal Sb and obtaining the difference therebetween. The specific circuit configuration of the detection circuit 123 will be described later in detail.

The band pass filter circuit 124 generates a filter signal Sd by removing both low frequency components and high-frequency components superimposed in the detection signal Sc and outputs it to a subsequent stage. Incidentally, it is desirable to set a band pass frequency of the band pass filter circuit 124 to about 0.6 to 4.0 Hz.

The amplifier circuit 125 generates an output signal Se by amplifying the filter signal Sd by a predetermined gain and outputs it to the control unit 13 of a subsequent stage.

The reference voltage generation circuit 126 generates a reference voltage VREF (=AVDD 2) by dividing a power supply voltage AVDD by two (2) and supplies it to each unit of the filter unit 12.

With the filter unit 12 of the exemplary configuration, it is possible to detect the pulse wave of an examinee with high accuracy when the examinee is not only taking a rest but also when moving (e.g., walking, jogging, running, etc.), since it can suitably remove the noise due to the body movement of the examinee.

Furthermore, in the filter unit 12 of the exemplary configuration, since the TIA 121, the buffer circuit 122, the detection circuit 123, the band pass filter circuit 124 and the amplifier circuit 125 all operate at the reference voltage VREF (=AVDD/2) as the center value, an output signal Se of the filter unit 12 has a waveform whose amplitude fluctuates up and down with respect to the reference voltage VREF. Accordingly, with the filter unit 12 of the exemplary configuration, it is possible to prevent the saturation of the output signal Se (e.g., the output signal Se is prevented from adhering to the power supply voltage AVDD or the ground voltage GND) and correctly detect pulse wave data.

In addition, with the filter unit 12 of the exemplary configuration, it is possible to accurately perform a pulse wave measurement (a pulse rate measurement) even in an environment for outdoor activities due to the detection processing of the difference between the upper and lower envelopes by the detection circuit 123. This will be described later in more detail.

### <TIA>

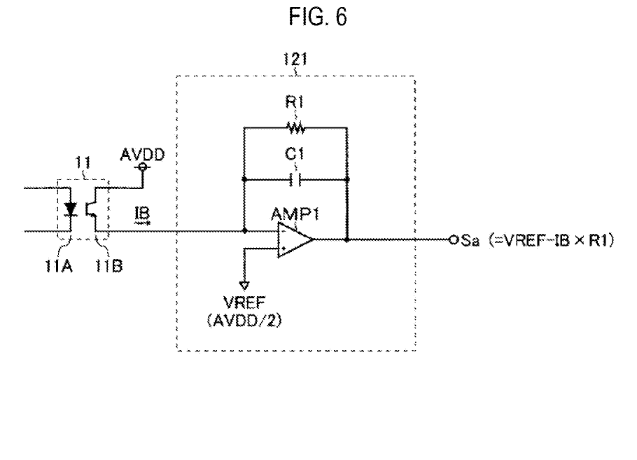

FIG. 6 shows a circuit diagram illustrating an exemplary configuration of a trans-impedance amplifier (TIA) 121. The TIA 121 of the exemplary configuration includes an operational amplifier AMP 1, a resistor R1, and a capacitor C1. A non-inverting input terminal (+) of the operational amplifier AMP1 is connected to an apply terminal of a reference voltage VREF (=AVDD/2). An inverting input terminal (−) of the operational amplifier AMP1 is connected to an emitter of a photo diode 11B. A collector of the photo diode 11B is connected to an apply terminal of a power supply voltage AVDD. An output terminal of the operational amplifier AMP1 corresponds to an output terminal of the voltage signal Sa. The resistor R1 and capacitor C1 are connected in parallel to each other between the inverting input terminal (−) and the output terminal of the operational amplifier AMP1.

In the TIA 121 of the exemplary configuration, a current signal TB flows through a current path from the inverting input terminal (−) of the operational amplifier AMP1 to an output terminal of a voltage signal Sa via the resistor R1. Accordingly, a voltage obtained by adding a voltage signal Sa to a voltage across the resistor R1 (=Sa+TB×R1) is applied to the inverting input terminal (−) of the operational amplifier AMP1. Meanwhile, the operational amplifier AMP1 having an imaginary short-circuit state between a non-inverting input terminal (+) and an inverting input terminal (−) produces an output signal Sa. Thus, a voltage signal Sa produced by the TIA 121 becomes a voltage value obtained by subtracting a voltage across the resistor R1 from the reference voltage VREF (=VREF−TB×R1).

Namely, the larger the current signal TB (corresponding to the light reception amount in the phototransistor 11B) flowing through the resistor R1 becomes, the lower the voltage signal Sa becomes. On the contrary, the smaller the current signal TB becomes, the higher the voltage signal Sa becomes. It is possible to arbitrarily adjust the gain of the TIA 121 by changing the resistance value of the resistor R1.

### <Detection Circuit>

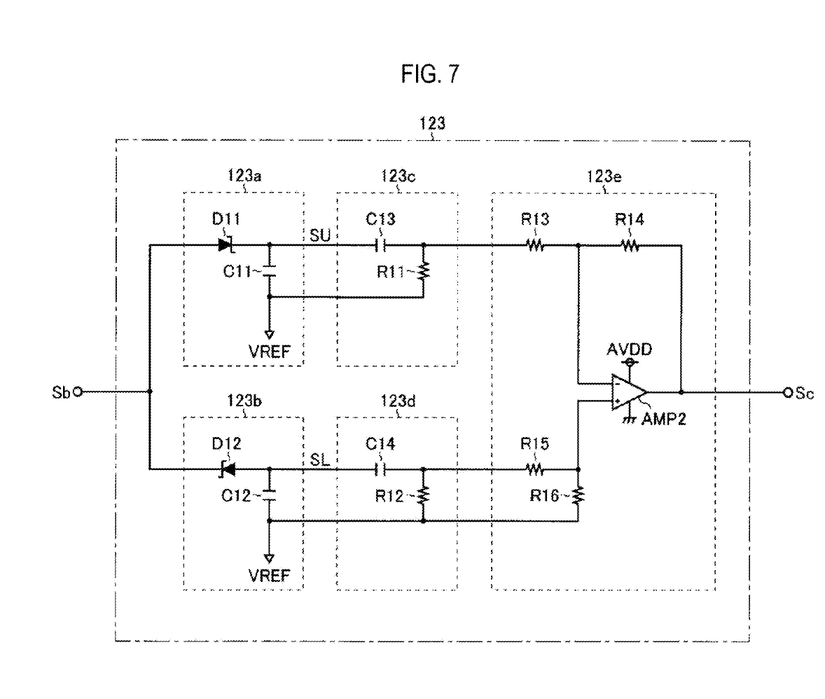

FIG. 7 shows a circuit diagram illustrating an exemplary configuration of a detection circuit 123. The detection circuit 123 of the exemplary configuration includes an upper envelope detector unit 123a, a lower envelope detector unit 123b, a level shifter unit 123c, a level shifter unit 123d, and a differential amplification unit 123e.

The upper envelope detector unit 123a is a circuit block that generates an upper envelope signal SU by extracting the upper envelope (i.e., the signal changes in the upper end side of pulses) of a buffer signal Sb (i.e., a voltage signal Sa input via a buffer 122). The upper envelope detector unit 123 a includes a diode D11 and a capacitor C11. An anode of the diode D11 is connected to an input terminal of the buffer signal Sb. A cathode of the diode D11 is connected to an output terminal of the upper envelope signal SU. A first terminal of the capacitor C11 is connected to an output terminal of the upper envelope signal SU. A second terminal of the capacitor C11 is connected to an apply terminal of the reference voltage VREF. If a bias point of the upper envelope signal SU is previously determined, a resistor may be connected to the capacitor C11 in parallel.

The lower envelope detector unit 123b is a circuit block that generates a lower envelope signal SL by extracting the lower envelope (i.e., the signal changes in the lower end side of pulses) of a buffer signal Sb. The lower envelope detector unit 123b includes a diode D12 and a capacitor C12. A cathode of the diode D12 is connected to an input terminal of the buffer signal Sb. An anode of the diode D12 is connected to an output terminal of the lower envelope signal SL. A first terminal of the capacitor C12 is connected to an output terminal of the lower envelope signal SL. A second terminal of the capacitor C12 is connected to a terminal for applying the reference voltage VREF.

The level shifter unit 123c is a circuit block that makes the signal level of the upper envelope signal SU matched to the input range of a differential amplification unit 123e. The level shifter unit 123c includes a capacitor C13 and a resistor R11. A first terminal of the capacitor C13 is connected to an output terminal of the upper envelope detector unit 123a. Both a second terminal of the capacitor C13 and a first terminal of the resistor R11 are connected to a first input terminal of the differential amplification unit 123e (i.e., an inverting input terminal). A second terminal of the resistor R11 is connected to an apply terminal of the reference voltage VREF.

The level shifter unit 123d is a circuit block that makes the signal level of the lower envelope signal SL matched to the input range of the differential amplification unit 123e. The level shifter unit 123d includes a capacitor C14 and a resistor R12. A first terminal of the capacitor C14 is connected to an output terminal of the lower envelope detector unit 123b. Both a second terminal of the capacitor C14 and a first terminal of the resistor R12 are connected to a second input terminal of the differential amplification unit 123e (i.e., a non-inverting input terminal). A second terminal of the resistor R12 is connected to an apply terminal of the reference voltage VREF.

Thus, it is possible to use a high pass filter operating at a reference voltage VREF as the level shifter units 123c and 123d, respectively.

The differential amplification unit 123e is a circuit block that generates a detection signal Sc by amplifying the difference between an upper envelope signal SU and a lower envelope signal SL input via each level shifter units 123c and 123d. The differential amplification unit 123e includes an operational amplifier AMP2 and resistors R13 to R16. A first terminal of a resistor R13 is connected to an output terminal of the level shifter unit 123c. Both a second terminal of the resistor R13 and a first terminal of a resistor R14 are connected to an inverting input terminal (−) of the operational amplifier AMP2. A second terminal of the resistor R14 is connected to an output terminal of the operational amplifier AMP2. A first terminal of a resistor R15 is connected to an output terminal of the level shifter unit 123d. Both a second terminal of the resistor R15 and a first of a resistor R16 are connected to a non-inverting input terminal (+) of the operational amplifier AMP2. A second terminal of the resistor R16 is connected to a terminal for applying the reference voltage VREF. An output terminal of the operational amplifier AMP2 is connected to an output terminal of the detection signal Sc.

### <Difference Detection Processing of Upper and Lower Envelopes>

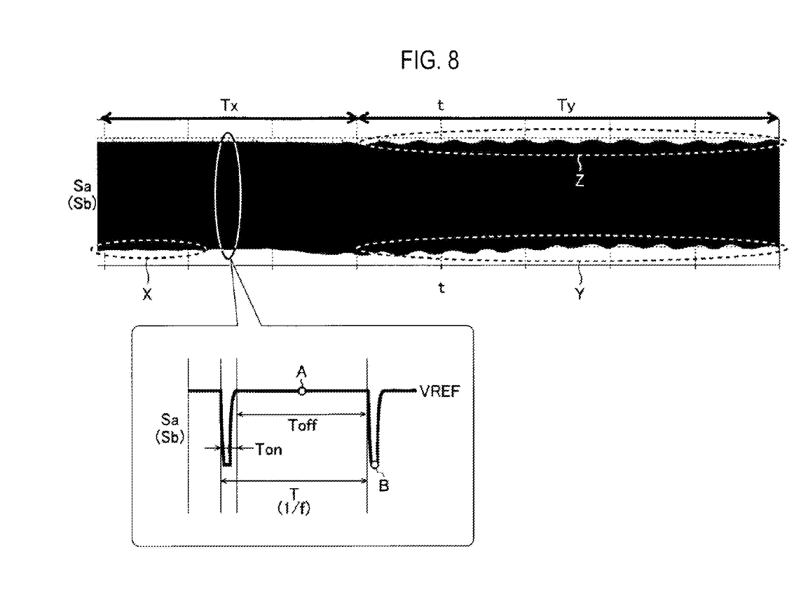

Next, referring to FIG. 8 to FIG. 10 as appropriate, the difference detection processing of upper and lower envelopes by a detection circuit 123 will be described in detail.

FIG. 8 shows a waveform diagram illustrating an example of a voltage signal Sa (consequently, a buffer signal Sb). FIG. 8 shows a waveform of a voltage signal Sa measured in a harsh sunny outdoor environment (e.g., the illuminance under sunshine being about 100,000 lux). Incidentally, a stationary period Tx is indicative of a period during which the examinee is stationary and an active period Ty is indicative of a period during which the examinee is on the move (e.g., jogging). In addition, an enlarged partial view of a voltage signal Sa during a stationary period Tx is shown in a lower frame of FIG. 8.

Incidentally, with respect to a pulse driving condition of a light emitting unit 11A, it is desirable to set a frame frequency to fall within a range from 50 to 1000 Hz (for example, f=128 Hz). Also, it is desirable to set an on-duty ratio Don (i.e., an occupancy ratio of the on-period Ton during a frame period (T=1/f)) to fall within a range from 1/200 to ⅛ (e. g., Don= 1/16).

As described above, a voltage signal Sa generated by the TIA 121 has a voltage value obtained by subtracting a voltage across the resistor R1 from the reference voltage VREF (=VREF−IB×R1). Herein, since the examinee is stationary during the stationary period Tx, the light sensor 1 is rarely lifted from the living body 2 (e.g., the wrist) and the incidence of extraneous light to the light receiving unit 11B is appropriately blocked. Accordingly, a body movement signal (i.e., a signal fluctuation component due to a body movement of the examinee) that is superimposed on a voltage signal Sa is sufficiently small. The voltage signal Sa obtained by the TIA 121 during a turn off-period Toff of the light emitting unit 11A, as represented by point A in the enlarged partial view, almost coincides with a reference voltage VREF (e.g., an off-voltage signal Sa@A).

Namely, during a stationary period Tx, the voltage signal Sa obtained by the TIA 121 during a turn on-period Ton of the light emitting unit 11A (e.g., an on-voltage signal Sa@B) has a voltage value that substantially fluctuates only according to the examinee's pulse rate (see the reference numeral X). Accordingly, as long as a pulse wave measurement is performed only during a stationary period Tx, it is also sufficiently possible to obtain pulse wave data (i.e., an output signal Se) of the examinee by extracting only the lower envelope of the voltage signal Sa (i.e., the signal changes of the on-voltage signal Sa@B).

On the other hand, since the examinee is moving during an active period Ty, the light sensor 1 is easily lifted from the living body 2 (e.g., the wrist) due to a body movement (such as arm swinging and landing impact) and the incidence of extraneous light to the light receiving unit 11B can't be sufficiently blocked. In particular, in a harsh sunny outdoor environment, a body movement signal that is superimposed on a voltage signal Sa has a non-negligible size.

In other words, during the active period Ty, the voltage value of the on-voltage signal Sa@B is fluctuated by the body movement of the examinee as well as the pulse rate of the examinee (see the reference numeral Y). Accordingly, if a pulse wave measurement is performed in an active period Ty as well as a stationary period Tx, it is impossible to obtain pulse wave data (i.e., an output signal Se) of the examinee with high accuracy simply by extracting only the lower envelope of the voltage signal Sa (i.e., the signal changes of the on-voltage signal Sa@B).

Herein, the inventors of the present disclosure observe that a body movement signal is expressed as an upper envelope of a voltage signal Sa (i.e., the signal changes in an off-voltage signal Sa@A) (see the reference numeral Z). As a result of intensive study, they obtained a novel idea that it is necessary to extract an upper envelope and a lower envelope of the voltage signal Sa and perform a difference detection processing therebetween, in order to obtain pulse wave data (i.e., an output signal Se) of the examinee with high accuracy by canceling the influence of the body movement signal during outdoor activities.

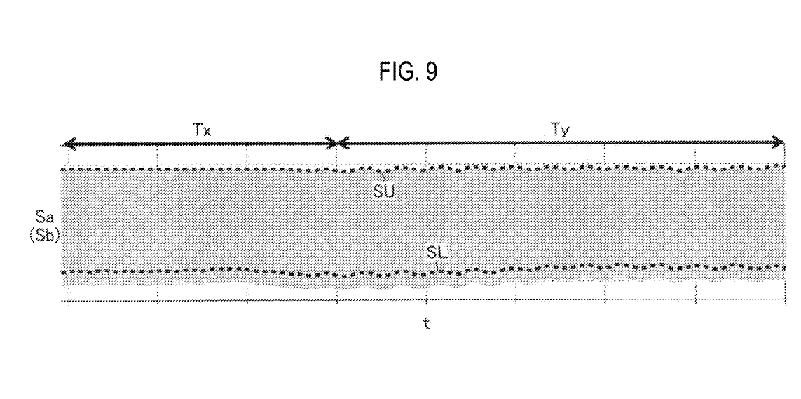

FIG. 9 shows a waveform diagram illustrating an example of an upper envelope signal SU and a lower envelope signal SL. For convenience of illustration, FIG. 9 shows a voltage signal Sa (or a buffer signal Sb) as represented by half-tone dot meshing. The upper envelope signal SU and the lower envelope signal SL are represented to be superimposed on the voltage signal Sa.

The upper envelope signal SU corresponds to a body motion signal as mentioned above. Accordingly, a signal value of the upper envelope signal SU becomes almost a fixed value (i.e., a reference voltage VREF) during a stationary period Tx and a variable value that varies according to the body movement of an examinee during an active period Ty.

Meanwhile, the lower envelope signal SL corresponds to a pulse wave signal on which a body movement signal is superimposed. Accordingly, it is possible to remove a body movement signal superimposed on a pulse wave signal by performing a difference processing between the upper envelope signal SU and the lower envelope signal SL.

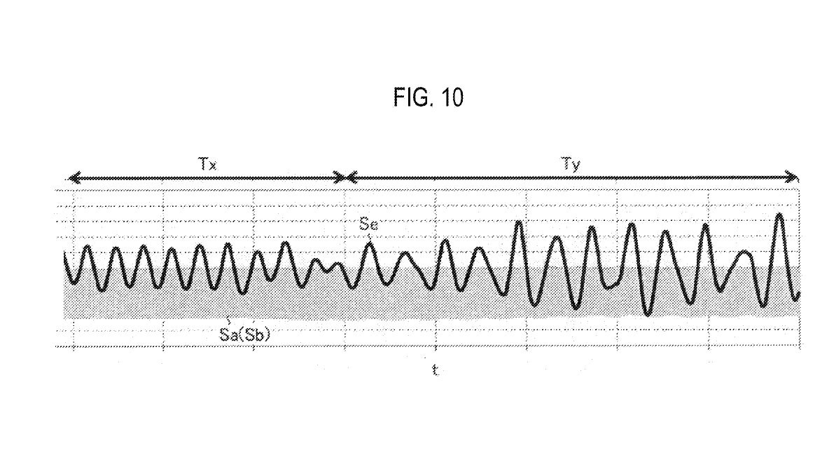

FIG. 10 shows a waveform diagram illustrating an example of an output signal Se obtained by a difference detection processing between the upper envelope and the lower envelope. It should be understood that an amplitude of the output signal Se falls within a predetermined range in any period of the stationary period Tx or the active period Ty without being very much affected by the pulse wave measuring situation (whether an examinee is stationary or moving). Consequently, it means that a body movement signal superimposed on the pulse wave signal has been properly removed by the difference detection processing between the upper envelope and lower envelope.

### <Evaluation Results>

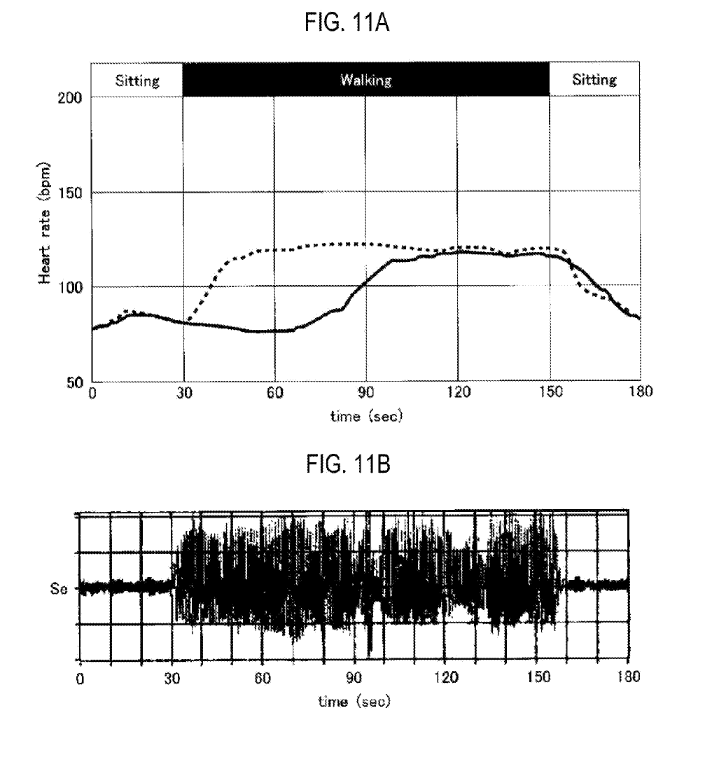

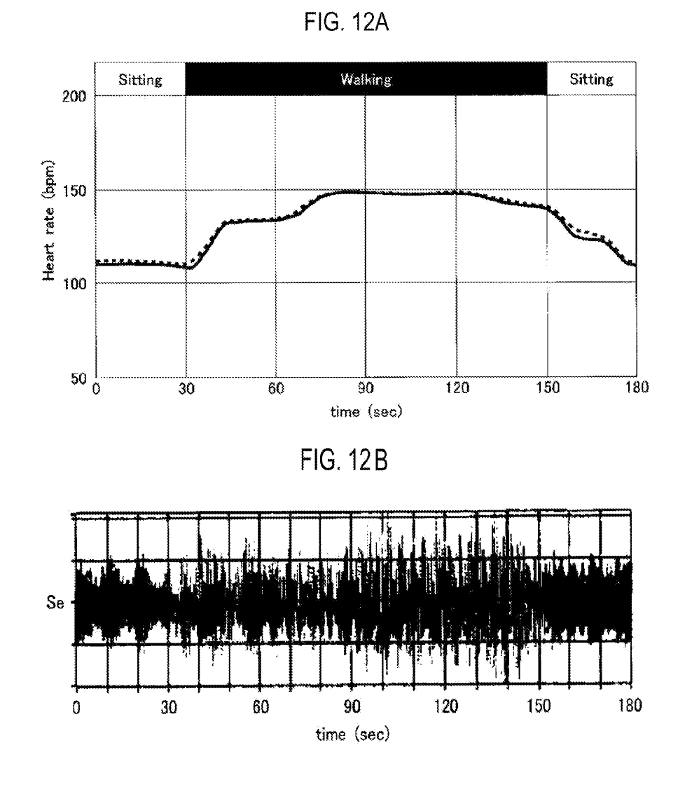

FIG. 11A and FIG. 11B are views illustrating an example of a first measurement (i.e., detection of a lower envelope only) in a harsh sunny outdoor environment (e.g., the illuminance under sunshine being about 100,000 lux) and FIG. 12A and FIG. 12B are views illustrating an example of a second measurement (i.e., detection of the difference between upper and lower envelopes) in the same environment. In addition, in FIG. 11A and FIG. 12A, the time change of a heart rate (e.g., beats per minute (bpm)) is depicted (as represented by a solid line for a photoelectric pulse wave sensor and a dashed line for a piezoelectric pulse wave sensor for reference) and in FIG. 11B and FIG. 12B, a waveform of the output signal Se is depicted.

As shown in FIG. 11A and FIG. 11B, if only a detection of the lower envelope is performed, the measurement result (i.e., pulse rate) during a stationary (e.g., seated) period almost coincides with the reference but the front half of the measurement result during an active period (e.g., walking) deviates from the reference. Also, if only the detection of the lower envelope is performed, it is necessary to set a gain of the output signal Se to be low to prevent the saturation of the measurement made during an active period, which is susceptible to body movements. As a result, even when performing a measurement during a stationary period less susceptible to body movements, the amplitude of the output signal becomes unnecessarily small and this causes a decrease of measurement accuracy.

Meanwhile, as shown in FIG. 12A and FIG. 12B, if a difference detection between an upper envelope and a lower envelope is performed, the measurement result (i.e., pulse rate) coincides with the reference during any of the stationary period and the active period. In addition, if the difference detection between an upper envelope and a lower envelope is performed, it is unnecessary to decrease a gain of the output signal Se since no significant change in the amplitude of the output signal Se occurs between the stationary period and the active period. Since it is possible to set the amplitude of the output signal Se to be sufficiently large, an increase of measurement accuracy in the active period as well as the stationary period occurs.

### <Consideration of Output Wavelength>

In an experiment, when a wavelength output from the light emitting unit is λ1 (Infrared: 940 nm), λ2 (Green: 630 nm) and λ3 (blue: 468 nm) and an output intensity of the light emitting unit (a driving current value) is changed to 1 mA, 5 mA and the 10 mA, behaviors in the pulse wave sensor of a so-called reflection type were investigated. As a result, since an absorption coefficient of the oxygenated hemoglobin HbO₂ increases and peak intensity of a pulse wave measurement becomes large in a visible light region where a wavelength is about 600 nm or small, it is understood that it is relatively easy to obtain a waveform of a pulse wave.

It is noted that, for a pulse oximeter to detect an oxygen saturation of arterial blood, a wavelength (e.g., before and after 700 nm) in a near infrared region where a difference between the absorption coefficient (i.e., solid line) of the oxygenated hemoglobin HbO₂ and the absorption coefficients of a deoxygenated hemoglobin Hb (i.e., dashed line) is a maximum has been widely used as a wavelength output from the light emitting unit generally. However, in experimental results as mentioned above, it is desirable to use a visible light region where a wavelength is 600 nm or smaller as a wavelength output from the light emitting unit, when using a pulse wave sensor (in particular, when using a pulse wave sensor of a so-called reflection type).

Only, when detecting both a pulse wave and a blood oxygen saturation using a single light sensor, it is also possible to use a wavelength in a near infrared region similar to a conventional case.

### <Other Modifications>

With respect to the configuration of various embodiments disclosed herein, in addition to the embodiments as described above, various modifications can be made without departing from the scope of the disclosure. While certain embodiments have been described, these embodiments have been presented by way of example only, and are not intended to limit the scope of the disclosures. Indeed, the novel methods and apparatuses described herein may be embodied in a variety of other forms; furthermore, various omissions, substitutions and changes in the form of the embodiments described herein may be made without departing from the spirit of the disclosures.

It is possible to use various disclosures disclosed herein as a technique to enhance the convenience of a pulse wave sensor and a sleep sensor. It is considered that the disclosures can be applied to various fields such as a healthcare support device, a gaming device, a music device, tools for communications with a pet and a drowsiness prevention apparatus of the vehicle's driver.

According to the present disclosure, it is possible to provide a pulse wave sensor capable of performing an accurate pulse wave measurement even in outdoor activities environment (e.g., pulse measuring).

What is claimed is:

1. A pulse wave sensor comprising:
an optical sensor unit configured to irradiate a living body with light emitted from a light emitting unit and detect light reflected from or transmitted through the living body with a light receiving unit to generate a current signal in accordance with a light reception intensity;
a pulse driving unit configured to turn on or off the light emitting unit at a predetermined frame frequency and a predetermined duty rate;
a current/voltage conversion circuit configured to convert the current signal into a voltage signal; and
a detection circuit configured to extract an upper envelope and a lower envelope of the voltage signal and obtain a difference therebetween to generate a detection signal.

2. The pulse wave sensor of claim 1, wherein the detection circuit includes:
an upper envelope detector unit configured to extract an upper envelope signal from the voltage signal;
a lower envelope detector unit configured to extract a lower envelope signal from the voltage signal; and
a differential amplification unit configured to obtain a difference between the lower envelope signal and the upper envelope signal to generate the detection signal.

3. The pulse wave sensor of claim 2, wherein the upper envelope detector unit includes:
a first diode including an anode connected to an input terminal for the voltage signal and a cathode connected to an output terminal for the upper envelope signal; and
a first capacitor including a first terminal connected to an output terminal for the upper envelope signal and a second terminal connected to a terminal for applying a reference voltage.

4. The pulse wave sensor of claim 2, wherein the lower envelope detector unit includes:
a second diode including a cathode connected to an input terminal for the voltage signal and an anode connected to an output terminal for the lower envelope signal; and
a second capacitor including a first terminal connected to an output terminal for the lower envelope signal and a second terminal connected to a terminal for applying a reference voltage.

5. The pulse wave sensor of claim 2, wherein the detection circuit further includes
a level shifter unit configured to adjust a signal level of the upper envelope signal and the lower envelope signal to respectively match an input range of the differential amplification unit.

6. The pulse wave sensor of claim 5, wherein the level shifter unit is a high pass filter.

7. The pulse wave sensor of claim 1, wherein the current/voltage conversion unit is a trans-impedance amplifier.

8. The pulse wave sensor of claim 1, further including a band pass filter circuit configured to remove both low-frequency components and high-frequency components superimposed on the detection signal to generate a filtered signal.

9. The pulse wave sensor of claim 8, further including an amplifier circuit configured to amplify the filtered signal by a predetermined gain to generate an output signal.

10. The pulse wave sensor of claim 1, wherein a wavelength output from the light emitting unit falls within a visible light region where a wavelength is 600 nm or smaller.
